(19) United States
(12) Patent Application Publication
(10) Pub. No.: US 2010/0074873 A1
(43) Pub. Date: Mar. 25, 2010

(54) Live bacteria product

(76) Inventor: James B. Watson, Pierce, NE (US)

Correspondence Address: THOMTE LAW OFFICE, L.L.C., 2120 S. 72ND STREET, SUITE 1111, OMAHA, NE, 68124, US

(21) Appl. No.: 12/592,615

(22) Filed: Nov. 30, 2009

Related U.S. Application Data
(63) Continuation-in-part of application No. 12/287,236, filed on Oct. 7, 2008.
Application No. 12/287,236 is a continuation-in-part of application No. 10/853,346, filed on May 25, 2004, now abandoned.

(51) Int. Cl. A61K 35/74; C12N 1/20

(52) U.S. Cl. 424/93.4; 435/252.1

(57) ABSTRACT

A live bacteria product including dormant viable bacteria suspended in a liquid carrier. The liquid carrier is sufficiently devoid of moisture so that substantially all of the bacteria will remain in a dormant state for several months or until applied to the target host. The liquid carrier has a moisture content of less than one percent and preferably has a moisture content of less than one-tenth of one percent. The carrier contains mineral oil and a suspension agent and may also include an adsorbent. The suspension agent may be polymers such as synthetic polymers. The product is stored and shipped in a plastic bag and is sprayed onto its target host or the like. The moisture and pH of the target host then activates the bacteria.

### CROSS-REFERENCE TO RELATED APPLICATION

This is a continuation-in-part application of Applicant's earlier application Ser. No. 12/287,236, filed Oct. 7, 2008, entitled “LIVE BACTERIA PRODUCT,” which was a continuation-in-part application of Ser. No. 10/853,346, filed May 25, 2004, entitled “LIVE BACTERIA PRODUCT”.

### BACKGROUND OF THE INVENTION

1. Field of the Invention

This invention relates to a live bacteria product and more particularly to a live bacteria product wherein live bacteria, that are in a dormant state, are suspended in a clear liquid fluid carrier or medium that can be easily pumped and will stay in suspension for the life of the product. More particularly, the carrier, while being in a liquid state, is sufficiently devoid of moisture to prevent the bacteria from becoming activated until the product is applied to feeds, forages, or directly onto animals such as livestock or the like. Hereinafter, feeds, forages, animals, livestock or the like will be referred to as “the target host”. Hereinafter, livestock will refer to cattle, horses, chickens, turkeys, hogs or the like.

2. Description of the Related Art

Live bacteria formulations are frequently applied to forage products, feed, etc., to enhance fermentation and/or digestion. The prior art products fall generally into two categories which are either a dry soluble powder form or a dry granular non-soluble form. The dry granular products are directly applied to its target host through a metering device such as a Gandy box, usually at a rate of 4 to 16 ounces per ton of forage treated. The dry soluble products must first be hydrated with water, then applied to its target host within a 48-hour time period post-hydration with application made through a liquid pump system at the rate of 37 grams to 1 gallon per ton of forage.

The dry granular products of the prior art, even if packaged in plastic pails, will sometimes come into direct contact with ambient air during manufacturing and processing or upon simply opening the product lid with the ambient air containing moisture or humidity. The moisture or humidity will activate the dormant bacteria upon contact which in turn causes the bacteria to inadvertently live and die before it can be applied to the forage. The value of the product will then be a total loss. The limestone carriers of the prior art products will carry minute traces of moisture, and such moisture severely reduces the product's ability to sustain an adequate shelf life. If the prior art product is a soluble powder product, it will also need to be hydrated before application. The hydration immediately activates all of the dormant bacteria and the producer has to apply the entire mixture within a 48-hour time frame or before since the bacteria will naturally deplete its food source and die, creating a total loss of the product value.

Further, the prior art products on the market today must be either removed from their original shipping container or physically poured into a dry applicator prior to application, or they must be contaminated with water, reducing their shelf life to less than 48 hours, post-contamination. Additionally, the prior art dry granular products have a tendency of sorting during shipment. This shakes any fines to the bottom of the shipping container leaving the either larger or lighter material to work its way to the top. Since the bacteria attach themselves to these particles, they may also migrate with these particles. Other dry soluble powder products may settle to the bottom of the applicator tank upon hydration. This will depend greatly on the product's rate of dilution. As application rates decrease to meet the producer's ongoing needs of hauling less product to the field, the manufacturers must in turn make their products more condensed to meet these needs. Such limits the solubility of the product.

The dry granular products of the prior art require a significant percentage of the product to be applied to the material which results in uniform coverage becoming difficult. Dry soluble powders mixed with water carriers have attempted to achieve low inclusion rates for some time. However, such products result in droplets which are very large in their molecular size and weight resulting in that there are actually few droplets being delivered per treatment, an example being one ton of forage treated with only ten large droplets so uniform coverage is poor.

One prior art reference is U.S. Pat. No. 4,161,397 issued Jul. 17, 1979 to Bellet et al. In the Bellet et al. composition, it appears that 99% of the bacteria die within one day and the other 1% of the bacteria die within 20 days. Further, in Bellet et al., the composition is applied to seeds which have fungicide thereon thereby making the treated seeds inappropriate for feeding to livestock or the like. As will be pointed out hereinafter, Applicant's product is sufficiently devoid of moisture so that substantially all the bacteria remain alive but in a dormant state for several months.

### SUMMARY OF THE INVENTION

A live bacteria product is disclosed wherein the live bacteria are in a dormant state and are suspended in a clear liquid fluid carrier or medium that may easily be pumped and will stay in suspension for the life of the product. More particularly, the carrier or medium for the live bacteria is comprised of a substantially moisture-free liquid which is comprised of oil such as mineral oil or processed oil obtained from animal, vegetable or petroleum origins. The oil medium has a moisture content of less than one percent and preferably has a moisture content of less than one-tenth of one percent. In some cases, the carrier will also have a hydrophilic molecular sieve adsorbent (moisture scavenger) included therein.

The product of this invention is preferably applied to the target host at a micro-treatment rate of 2 to 28 grams per ton of target host being treated. In some cases, more or less percentages may be utilized. The live bacteria, by being suspended in the substantially moisture-free carrier, will remain dormant until coming into contact with the target host at which time the moisture in the target host will cause the bacteria to be activated. The product of this invention is packaged in controlled shipping vessels such as polyurethane bags that reduce the risk of moisture contamination of any kind. The product is sold and shipped directly to the producer who will then take the collapsible bag of product and hook it directly to an applicator which applies the product to the target host. The target host may be forage, feed or the like. In some cases, the product may be sprayed directly onto animals such as chicks who will ingest the product while grooming themselves.

It is therefore a principal object of the invention to provide an improved live bacteria product.

Yet another object of the invention is to provide a live bacteria product wherein live bacteria, that are in a dormant state, are suspended in a clear liquid fluid carrier or medium that can be easily pumped and will stay in suspension for the life of the product.

Still another object of the invention is to provide a live bacteria product which is substantially free of moisture to prevent the bacteria from becoming activated for several months or until the product is applied to the target host.

Still another object of the invention is to provide a live bacteria product which may be applied to a target host at an extremely small rate.

Still another object of the invention is to provide a live bacteria product wherein the carrier or medium therefore is comprised of a substantially moisture-free liquid comprised of mineral oil and polymers other than the bacteria.

Still another object of the invention is to provide a live bacteria product wherein the carrier or medium therefore has a moisture content of less than one percent and preferably less than one-tenth of one percent.

Still another object of the invention is to provide a live bacteria product which will not be activated until coming into contact with moisture associated with the target host.

Yet another object of the invention is to provide a live bacteria product which may be applied to livestock forage or feed at a preferred micro-treatment rate.

A further object of the invention is to provide a live bacteria product which is packaged in such a way to limit its exposure to possible contamination from outside sources, such as moisture-laden air or fluid water of any kind.

These and other objects will be apparent to those skilled in the art.

### BRIEF DESCRIPTION OF THE DRAWINGS

FIG. 1 is a graph referred to as FIG. 1;

FIG. 2 is a graph referred to as FIG. 2; and

FIG. 3 is a graph referred to as FIG. 3.

### DETAILED DESCRIPTION OF THE INVENTION

In this invention, conventional dormant viable harmless bacteria are mixed with a substantially moisture-free liquid carrier or medium. The liquid carrier has a moisture content of less than one percent and preferably has a moisture content of less than one-tenth of one percent. In general, the live bacteria used in the product of this invention will be any lactic acid producing bacteria that is permissible for use in animal related products. The United States Food and Drug Administration (FDA) and The American Association of Feed Control Officials (AAFCO) have published a list of the microorganism species which are “generally recognized as safe” (GRAS) for use in direct-fed microbial products. Table I hereinbelow lists presently approved bacteria for use with animal related products.

TABLE I
36.14 -- DFM Microorganisms under the “GRAS” Status

| | |
|---|---|
| Aspergillus niger | Lactobacillus curvatus |
| Aspergillus oryzae | Lactobacillus delbruekii |
| Bacillus coagulans | Lactobacillus fermentum |
| Bacillus lentus | Lactobacillus helveticus |
| Bacillus licheniformis | Lactobacillus lactis |
| Bacillus pumilus | Lactobacillus plantarum |
| Bacillus subtilis | Lactobacillus euterii |
| Bacteroides amylophilus | Leuconostoc mesenteroides |
| Bacteroides capillosus | Pediococcus acidilacticii |
| Bacteroides ruminocola | Pediococcus cervisiae |
| Bacteroides suis | Pediococcus pentosaceus |
| Bifidobacterium adolescentis | Propionibacterium freudenreichii |
| Bifidobacterium animalis | Propionibacterium shermanii |
| Bifidobacterium bifidum | Saccharomyces cerevisiae |
| Bifidobacterium infantis | Entercococcus cremoris |
| Bifidobacterium longum | Entercococcus diacetylactis |
| Bifidobacterium thermophilum | Entercococcus faecium |
| Lactobacillus acidolphilus | Entercococcus intermedius |
| Lactobacillus brevis | Entercococcus lactis |
| Lactobacillus buchneri | Entercococcus thermophilius |
| Lactobacillus bulgaricus | Lactobacillus cellobiosus |
| Lactobacillus casei | |

Preferably, the moisture-free liquid carrier is comprised of a product marketed under the trademark “SYNERGEL®” manufactured by Penreco of 138 Petrolia Street, Karns City, Pa. 16041-9799. The product is generally referred to as a gelled mineral oil, reference number 1004-100, having a Brookfield viscosity of 1242 cPs. The carrier product is clear, odorless and is insoluble in water. Other types of oils such as processed oils obtained from animal, vegetable or petroleum origins may be utilized provided they have a moisture content of less than one percent and preferably a moisture content of less than one-tenth of one percent. The carrier also includes a suspension agent such as synthetic polymers. Other suspension agents such as COBISAL™ (polymer fiber) may also be used from time to time to aid or reduce the possibility of the settling of the bacteria therein. The product of this invention is designed to only activate the bacteria at the physical point of contact with the target host. Upon contact, the target host ambient moisture content will activate (i.e., initiate the life cycle thereof) the dormant bacteria found within the liquid microbial medium or carrier. The preferred liquid microbial medium or carrier of this invention has undergone a special production process developed by Penreco. The process consists of first passing the mineral oil through an advanced filtration system which specifically targets and absorbs any ambient moisture. Secondly, the mineral oil is heated and polymers are added to increase the oil viscosity and suspension qualities. Thirdly, the oil is packaged into controlled shipping vessels that reduce the risk of moisture contamination of any kind.

Upon receipt of the packaged carrier, applicant then preferably adds a moisture scavenger product such as a hydrophilic molecular sieve adsorbent as an insurance program. The adsorbent may comprise a natural or synthetic zeolite consisting of crystalline metal aluminosilicate, alkali metal aluminosilicate or sodium aluminosilicate. Finally, whatever bacteria are needed for a particular purpose will then be added. The final blend is then packaged in moisture and ultraviolet retardant containers such as collapsible polyurethane bags, very similar to I.V. bags. The product is then sold and shipped directly to the retailer and/or end user. The end user will then take the collapsible bag of liquid microbial blend and hook it directly to an applicator so that extremely small droplets are created which are sprayed upon the target host.

The invention described herein provides the ability to inoculate other products with dormant live bacteria, by means of a light spraying application. Whereas the bacteria is protected with the oil/polymer blend allowing the host bacteria to survive longer in a non-favorable environment, the coating of the bacteria cell walls with the oil/polymer covering provides a physical chemical moisture barrier. The above provides the ability to permit the live bacteria to be introduced onto a pelleted feed without immediately inadvertently activating the oil/polymer/host bacteria through ambient moisture contamination from the feed itself.

The product of this invention permits it to be applied to its target host at a preferred micro-treatment rate of 2 to 28 grams per one ton (2,000 pounds) of forage or the material treated (with no additional water or carriers needed). In some cases, higher or lower ratios may be employed. Such micro-treatment is substantially less than prior art products that have treatment rates that range from 37.88 grams to 8.3453 pounds per one ton of forage treated.

The product of this invention and its packaging limits its exposure to possible contamination from outside sources, such as moisture-laden air or fluid water of any kind. The product of this invention is designed to only activate at the physical point of contact with the target host. Upon contact with the target host, the change in pH and moisture activates the dormant bacteria.

To remain 100% alive, freeze-dried bacteria must be kept free of water activity, kept out of direct ultra violet light, and maintained in temperatures less than 110° F. The best place to meet all of these criteria is to keep the bacteria in liquid nitrogen or a freezer. If kept frozen, the bacteria will remain alive and viable for many years. Unfortunately, if you want to apply these bacteria to agricultural needs, the bacteria must be taken out of the freezer and put into other types of carriers. In doing so, the bacteria are inadvertently exposed to ambient water activity.

Manufacturers have used oils for their residual effect and as a carrier for the freeze-dried bacteria under the assumption that oil would protect the bacteria from ambient moistures, thereby extending the life span of the bacteria. However, when the bacteria are placed in a feed grade mineral oil, the bacteria quickly die off in about 30 days. Another noted problem is that the bacteria in oil-based carriers quickly settle out, since the bacteria are non-soluble in oil.

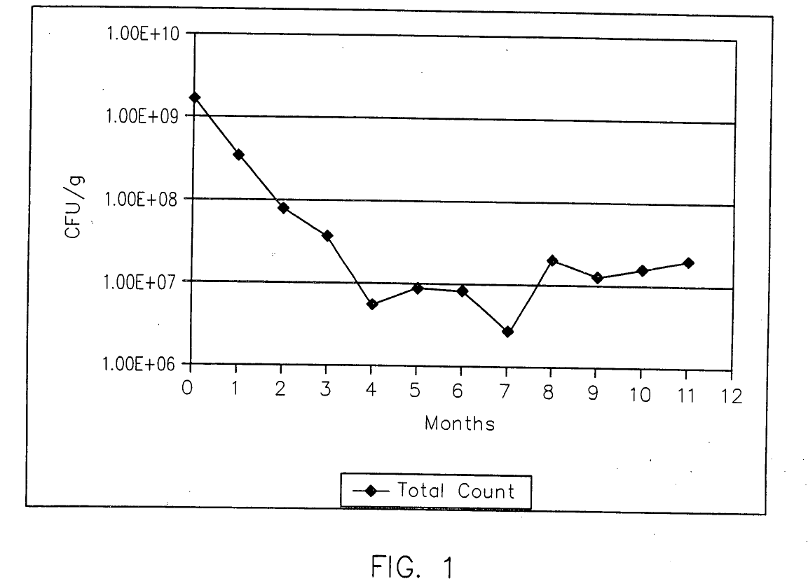

In developing the product described in this application, Applicant realized the problems with putting freeze-dried bacteria in an oil-based carrier. Testing showed that the bacteria in a feed grade mineral oil had a very short life span as seen in FIG. 1. In FIG. 1, a one-log die off represents a 90% die off of live bacteria. For example, (1.00 E 09-to-I.00 E 08)=a 90% loss of live bacteria. A two-log loss, for example, (1.00 E 09- to-1.00 E07)=a 99% loss of live bacteria. A three-log loss represents a 99.9% loss of live bacteria and so on.

FIG. 1 demonstrates the life span of freeze-dried Lactobacillius acidolphilus bacteria blended into a feed grade mineral oil. Please note that there was a 78.92% die off of the La bacteria in the first month, a 95.07% die off on the second month and a 97.78% total cumulative die off of the La bacteria upon the third month with a 99.67% die off on the fourth month.

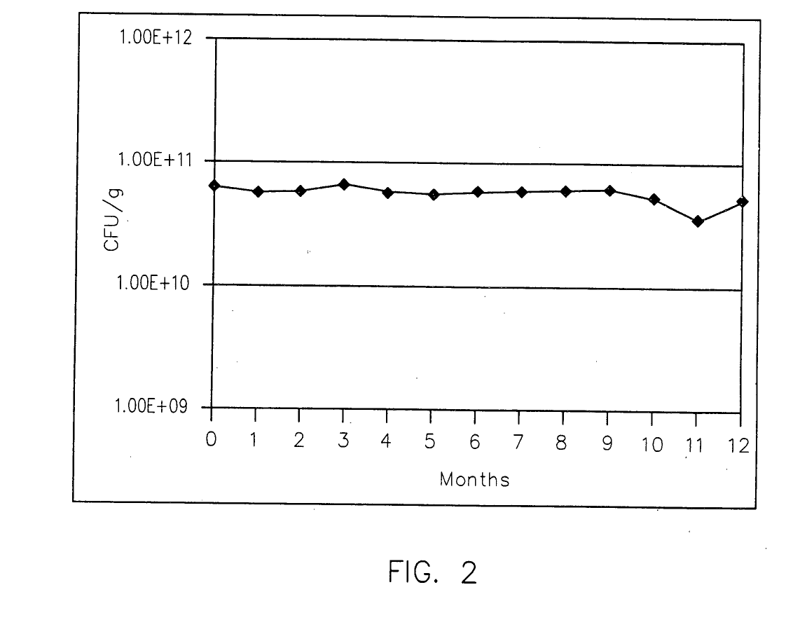

FIG. 2 illustrates a test of Lactobacillius acidolphilus bacteria blended into the carrier product described and claimed in this application and it can be seen that the bacteria in FIG. 2 lived for at least 12 months' time, at which time the test was discontinued.

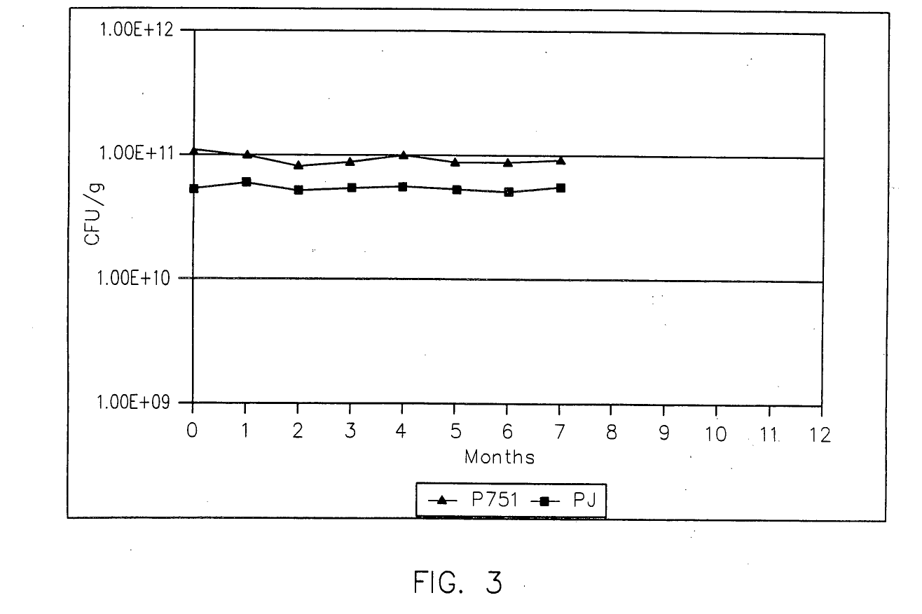

FIG. 3 is a currently ongoing experiment, scheduled for 12 months testing. In its seventh month of testing, FIG. 3 shows two bacteria being separately tested in the product described and claimed in this application. In the tests of FIG. 3, (P751) Pediococcus acidilaticii and (PJ) Pediococcus pentosaceus were still 100% alive at 7 months' time.

It can be seen from FIGS. 2 and 3, wherein the carrier of this application was utilized, the life span of the freeze-dried bacteria is greatly extended than that previously possible.

The composition of this invention coats the bacteria with a protective coating. When the composition is applied to livestock feed, the protective coating protects the bacteria from hydration for a period of time which is at least 40 days. Once the treated livestock feed is ingested by the target host, it is immediately subjected to a low intestinal pH environment. The low pH environment breaks down the protective coating on the bacteria which then becomes hydrated by the ambient moisture within the digestion system, thus causing the bacteria to begin their life cycle.

Thus it can be seen that the invention accomplishes at least all of its stated objectives.

1. A composition useful for treating livestock feed for ingestion by livestock, comprising:
a liquid suspension of dormant viable bacteria, oil and a suspension agent with the liquid suspension being sufficiently devoid of moisture so that substantially all the bacteria will remain in a dormant state for several months or until applied to the livestock feed;
said liquid suspension having a moisture content of less than one percent.

2. The composition of claim 1 wherein substantially all the bacteria will remain in a dormant state for at least seven months.

3. The composition of claim 1 wherein substantially all the bacteria will remain in a dormant state for at least twelve months.

4. The composition of claim 1 wherein the liquid suspension also includes a moisture scavenger.

5. The composition of claim 4 wherein the moisture scavenger comprises an adsorbent.

6. The composition of claim 4 wherein the moisture scavenger comprises zeolite.

7. The composition of claim 4 wherein the moisture scavenger is comprised of crystalline metal aluminosilicate, alkali metal aluminosilicate or sodium aluminosilicate.

8. The composition of claim 4 wherein the moisture scavenger comprises a hydrophilic molecular sieve adsorbent.

9. The composition of claim 1 wherein the bacteria is a lactic acid producing bacteria.

10. The composition of claim 1 wherein said oil is clear, odorless and insoluble in water.

11. The composition of claim 10 wherein said oil is a gelled mineral oil.

12. The composition of claim 11 wherein said gelled mineral oil has a Brookfield viscosity of 1242 cPs.

13. The composition of claim 1 wherein said liquid suspension has a moisture content of less than one-tenth of one percent.

14. A liquid carrier for live freeze dried bacteria to extend the life thereof for several months comprised of an oil which has been treated to remove moisture therefrom and which has a suspension agent added thereto to increase the viscosity and suspension qualities thereof with the oil having a moisture content of less than one percent.

15. The liquid carrier of claim 14 wherein the live freeze-dried bacteria is approved for use with livestock feed.

16. The liquid carrier of claim 14 wherein the live freeze-dried bacteria is approved for use on livestock.

17. The liquid carrier of claim 14 wherein the liquid carrier also includes a moisture scavenger.

18. The liquid carrier of claim 14 wherein said bacteria comprises Lactobacillus acidolphilus.

19. The liquid carrier of claim 14 wherein said bacteria comprises Pediococcus acidilacticii.

20. The liquid carrier of claim 14 wherein said bacteria comprises Pediococcus pentosaceus.

21. The liquid carrier of claim 14 wherein said bacteria is selected from the group consisting of:

| | |
|---|---|
| Aspergillus niger | Lactobacillus curvatus |
| Aspergillus oryzae | Lactobacillus delbruekii |
| Bacillus coagulans | Lactobacillus fermentum |
| Bacillus lentus | Lactobacillus helveticus |
| Bacillus licheniformis | Lactobacillus lactis |
| Bacillus pumilus | Lactobacillus plantarum |
| Bacillus subtilis | Lactobacillus euterii |
| Bacteroides amylophilus | Leuconostoc mesenteroides |
| Bacteroides capillosus | Pediococcus acidilacticii |
| Bacteroides ruminocola | Pediococcus cervisiae |
| Bacteroides suis | Pediococcus pentosaceus |
| Bifidobacterium adolescentis | Propionibacterium freudenreichii |
| Bifidobacterium animalis | Propionibacterium shermanii |
| Bifidobacterium bifidum | Saccharomyces cerevisiae |
| Bifidobacterium infantis | Entercococcus cremoris |
| Bifidobacterium longum | Entercococcus diacetylactis |
| Bifidobacterium thermophilum | Entercococcus faecium |
| Lactobacillus acidolphilus | Entercococcus intermedius |
| Lactobacillus brevis | Entercococcus lactis |
| Lactobacillus buchneri | Entercococcus thermophilius |
| Lactobacillus bulgaricus | Lactobacillus casei |
| Lactobacillus cellobiosus | |

22. The composition of claim 14 wherein said oil is clear, odorless and insoluble in water.

23. The liquid carrier of claim 14 wherein said oil is a gelled mineral oil.

24. The liquid carrier of claim 23 wherein said gelled mineral oil has a Brookfield viscosity of 1242 cPs.

25. The composition of claim 1 wherein said oil has a moisture content of less than one-tenth of one percent.

26. The composition of claim 1 wherein said suspension agent comprises synthetic polymers.
